(19) United States
(12) Patent Application Publication
(10) Pub. No.: US 2007/0224228 A1
(43) Pub. Date: Sep. 27, 2007

(54) Soap Bars Having Ultraviolet Radiation Protection

(76) Inventor: Clay J. Cockerell, Dallas, TX (US)

Correspondence Address: Louis C. Paul;Louis C. Paul & Associates, 730 5th Avenue, 9th Floor, New York, NY, 10019, US

(21) Appl. No.: 11/596,861

(22) PCT Filed: May 13, 2005
(86) PCT No.: PCT/US05/16632

§ 371 Date: Nov. 17, 2006

Related U.S. Application Data
(60) Provisional application No. 60/571,973, filed on May 18, 2004.

(51) Int. Cl. A61K 9/00; A61K 8/00; A61Q 1/00; A61K 8/02

(52) U.S. Cl. 424/401; 424/400; 424/59; 424/60

(57) ABSTRACT

The present invention is drawn to a soap bar that after the washing process leaves a residual deposit on a substrate that provides protection from ultraviolet radiation comprising (a) soap, a fatty carboxylic acid that has been saponified with either sodium hydroxide, potassium hydroxide, ammonium hydroxide, or mixtures thereof; (b) from greater than 10% to about 25% by weight red petrolatum; and (c) at least one surface-treated metal oxide. Optionally, compositions of the present invention may also include (d) at least one wetting agent having a log P value of greater than about 4.0 and/or (e) an organic sunscreen having a log P value of greater than about 4.0.

### CROSS-REFERENCE TO RELATED APPLICATIONS

This application claims priority to U.S. Provisional Application Ser. No. 60/571,973 filed May 18, 2004.

### STATEMENT REGARDING FEDERALLY-SPONSORED RESEARCH OR DEVELOPMENT

Not applicable.

### FIELD OF THE INVENTION

This invention relates to soap bars which are formulated to deposit on a substrate (e.g., the skin or hair) ultraviolet light absorbing/reflecting material that provides protection from ultraviolet radiation (“UVR”). It also relates to a process for treating skin, hair and other substrates with such a soap bar.

### BACKGROUND OF THE INVENTION

Soaps have long played an essential role in promoting health and hygiene. Soaps are water-soluble sodium or potassium salts of fatty acids. Generally, soaps are produced by one of two processes. In saponification, fats and oils are heated and reacted with a liquid alkali, commonly sodium hydroxide or potassium hydroxide (also referred to as lye). The reaction produces crude soap, water and glycerine. The earliest soaps were made by boiling lard or other animal fat together with potassium carbonate (potash) prepared by leaching wood ashes with water. In a second process, the ester bonds of fats and oils are cleaved (hydrolyzed) into fatty acids and glycerine. The resulting fatty acids are purified (by distillation) and then neutralized with an alkali to produce soap and water.

From early times, the use of soaps has been medically motivated. For example, the Greek physician Galen, recommended washing with soap as a prophylactic, particularly for preventing diseases of the skin. In the past century, as the relationship between cleanliness and hygiene became recognized, the widespread use of soaps was encouraged to stop the transmission of infectious diseases. Antibacterial soaps were developed to kill disease-causing germs. Deodorant soaps were commercialized to inhibit the growth of odor-causing bacteria. Illustrative of the antibacterial and bacterial soap bars are those sold by the Dial Corp.

While the ability to remove grease and oil (e.g., that adheres to skin and fabrics) remains the principal attribute by which soaps are measured, other properties have taken on added significance to consumers. These include lather quality (both in terms of lather volume and lather viscosity), mildness, skin after-feel, deodorancy, and use of natural and/or environmentally friendly ingredients. The ability to effectively deliver active ingredients to the skin during washing with a soap bar has become an increasingly important—but far from fully met—consumer need.

Skin cancer is the most prevalent of all cancers. Each year, over one million new cases of skin cancer are diagnosed in the United States. Melanoma accounts for approximately 3% of all cancers in the United States, and approximately 1% of all cancer deaths. Incidence rates of malignant melanoma are increasing by 4-8% per year. In many, if not most, instances, skin cancer is preventable—in large part, through the consistent and appropriate use of sunscreens.

In an effort to change behaviors and increase sunscreen use, the public health community has undertaken a wide range of activities to raise awareness of skin cancer and other adverse health consequences associated with unprotected exposure to UVR. These include, for example, the Choose Your Cover campaign by the Centers for Disease Control and the annual Melanoma Monday events organized by the American Academy of Dermatology. Despite the alarming statistics, changing undesirable health behaviors, and eliciting consistent and appropriate use of sunscreens among the public has proven an elusive goal.

In part, this may be attributable to the popular media, which continues to create positive associations between a tanned appearance and good health. Another explanation for the limited success of education comes from sociopsychology—changes in behavior are often slow to come and occur idiosyncratically, often depending on the motivation of individual patients to change. A more effective approach, one that is more immediately impactful, is needed. Suggestions have included mandating uses of sunscreen and sun-protective clothing (e.g., in children's activities) or through economic incentives (e.g., lowering taxes on sunscreens). Cockerell, C J, “The War Against Cancer: The Time is Now” Arch. Dermatol. Vol. 141:409 (April 2005).

Even if skin-protective behaviors were made “compulsory”, the issue of compliance would remain. Compliance has been the bugaboo of many efforts to improve health and safety. For example, early laws requiring use of seat belts in automobiles were dependent on an additional behavior by driver and passenger—actively buckling-up. The introduction of passive restraint devices (air bags) increased the level of protection without requiring further action on the part of car occupants. Regrettably, short of avoiding sun exposure, an analogous, “passive” approach to UVR protection does not currently exist. The next-best alternative may be to incorporate UVR protection into another common, widely-accepted, routine behavior—ideally, one that is performed on a daily basis.

From a public health perspective, widespread, regular use of soap is credited with having a significant impact on reducing the spread of infectious disease. Coupled with the social stigma attached to having an unclean appearance and/or malodor, washing with a soap formulated to deliver UVR-protecting ingredients can provide a common, routine behavior needed to increase use of sunscreens.

It has been a long-desired goal to make a soap bar that not only provides cleaning and sanitizing attributes but also deposits a sunscreen that provides protection from the deleterious effects of the sun. This goal has been elusive for two related reasons. First, sunscreens are typically formulated in delivery systems that contain oils (e.g., oil-in-water emulsions). The very act of washing the skin with soap results in removal of oily materials, including those that contain UVR-protective ingredients. Second, where a soap does deposit a sunscreen on the skin, it typically does so by leaving an unattractive, visible white film. Given the demands of a highly appearance-conscious consumer population, from a marketing and consumer acceptance standpoint, cosmetic and personal care products that leave a visible film on the skin are viewed as unacceptable.

Differing approaches to delivering sunscreens in a soap or detergent wash product have been described in the literature. U.S. Pat. No. 6,825,161 teaches a controlled delivery system that can be incorporated in soap bars to enhance deposition of active ingredients. The carrier system comprises substantially free-flowing, powder formed of solid hydrophobic, positively charged, nanospheres of encapsulated active ingredients that are encapsulated in moisture sensitive microspheres.

Encapsulation of sunscreens in a liquid personal wash compositions is also taught in U.S. Pat. No. 6,217,852. The liquid wash sunscreen composition according to the '852 patent comprises at least two encapsulated, time-release sunscreens—one organic, the second inorganic—in combination with either an ethoxyldiglycol or a carboxylate-based emollient. The latter two ingredients are taught to act as penetration enhancers. Example 19 of the '852 patent teaches an alkoxysilane surface-treated inorganic sunscreen (zinc oxide) at 5% by weight of the composition. (Unless otherwise noted, numeric values expressed as a percentage in the present application represent the percentage by weight of the ingredient based on the total weight of the composition.) The liquid wash composition described in Example 19 of the '852 patent contained white petrolatum at a concentration of 10%. Example 30 of the '852 patent teaches two alkoxysilane surface-treated inorganic sunscreens—17% titanium dioxide and 5% zinc oxide—in a composition containing 5% white petrolatum.

U.S. Pat. No. 6,762,158 teaches personal care compositions for delivery and/or deposition of active ingredients, including sunscreens. The disclosed composition comprise from about 10% to about 80% of at least two water dispersible components that when combined with at least a weight equivalent of water produces a uniform clear or a uniform hazy mixture. Compositions of the '158 patent are also taught to contain two liquid esters.

U.S. Pat. No. 6,043,204 teaches a body cleansing composition for providing protection against sunburn after rinsing. The '204 patent teaches a specific combination of at least two sunscreens—a paramethoxycinnamate ester and at least one other sunscreen selected from the group consisting of a 2-hydroxy-4-alkoxybenzophenone, a salicylic acid ester, octocrylene, zinc oxide and mixtures thereof. Compositions of the '204 patent are also taught to contain at least two surface active agents: (i) from 3 to 15% of an alkyl sulfate salt or an alkoxylated alkyl sulfate salt; and (ii) from 1 to 10% of a nonionic or zwitterionic surfactant, or from 1% to 10% of an anionic surfactant that is not an alkyl sulfate salt.

U.S. Pat. No. 6,699,824 teaches cleansing compositions comprising highly branched polyalphaolefins of a certain formula and certain high viscosity. Polyalphaolefins are synthetic hydrocarbons formed by condensation polymerization of an alphaolefin, in which methylene groups alternate with alkyl groups. In contrast, red petrolatum as used in the present invention is a non-synthetic product comprising branched and unbranched hydrocarbons, with a viscosity significantly below that taught in the '824 patent for polyalphaolefins.

U.S. Pat. No. 6,576,228 discloses a personal wash composition using two sunscreens—phenylbenzimidazole sulphonic acid, ferulic acid, anionic salts of these acids, and mixtures of these acids and their salts.

U.S. Pat. No. 6,362,146 teaches rinse-off liquid personal wash cleaning compositions, comprising an encapsulated sunscreen active, where the sunscreen is from 5% to 60% by weight of the capsule. The liquid wash according to the '146 patent is taught to contain a cationic polymeric deposition aid. Preferred polymer deposition aids are cationic derivatives of guar gum, more particularly polygalactomannan gum.

U.S. Pat. No. 5,989,536 discloses oil-in-water emulsion composition useful for personal cleansing and for depositing active ingredients, including sunscreens, on the skin. More particularly, the '536 patent teaches use of polymeric thickener, an alkoxylated ether, and a cationic ammonium salt in an oil-in-water emulsion. The active ingredients useful in the disclosed emulsion are taught to have a solubility parameter of from about 7 to about 13.

In 1999, Performance Brands of Plantation Florida marketed a liquid wash product under the tradename SoapScreen®. This product contained a combination of D organic sunscreens—octylmethoxycinnamate, octyl salicylate and oxybenzone.

There remains a long-felt, but as yet unmet need, for a soap bar that deposits a film after washing which is barely perceptible while at the same time substantive enough to provide a significant measure of protection from ultra-violet radiation using metal oxide sunscreens which remain longer on the surface of the skin and, because they cannot penetrate into the skin, are safer to use, especially in children. This need is met by the soap bar of the present invention. It has now been discovered that incorporating red petrolatum plays an essential role in the effective deposition of metal oxide sunscreen on the skin in soap bar without negatively affecting the cleansing properties of the soap (i.e., ability to remove dirt in oils).

It is therefore one object of the present invention to provide soap compositions that provide a protection from UVR to skin, hair or other substrates when applied during the washing process. It is another objective of the present invention to provide a process for cleaning skin, hair or other substrate and protecting it from deleterious effects of ultraviolet radiation with a composition of the present invention. Other objects of the present invention will become clear as one reads the disclosure hereinbelow.

### BRIEF DESCRIPTION OF THE INVENTION

The present invention is drawn to a soap bar comprising (a) soap, a fatty carboxylic acid that has been saponified with either sodium hydroxide, potassium hydroxide, ammonium hydroxide, or mixtures thereof; (b) from greater than 10% to about 25% by weight red petrolatum; and (c) at least one surface-treated metal oxide sunscreen selected from the group consisting of micronized zinc oxide surface-treated with alkoxysilane; micronized titanium dioxide surface-treated with alkoxysilane; micronized titanium dioxide surface-treated with (i) silica, alumina and dimethicone/methicone copolymer or (ii) alumina and dimethicone/methicone copolymer; and mixtures thereof. Optionally, compositions of the present invention may also include (d) at least one wetting agent having a log P value of greater than about 4.0 and/or (e) an organic sunscreen having a log P value of greater than about 4.0.

### DETAILED DESCRIPTION OF THE INVENTION

The present invention is directed to a soap bar that provides protection to a substrate (e.g., skin or hair) from ultraviolet radiation after a washing process comprising:
(a) a fatty soap conforming to the following structure:
   R¹—C(O)—O⁻M⁺
wherein;
   R¹ is alkyl having 9 to 17 carbon atoms;
   M is a counterion needed for charge balance and is selected from the group consisting of Na, K and NH₄;
(b) from greater than 10% to about 25% by weight of the total composition of red petrolatum;
(c) at least one surface-treated metal oxide sunscreen selected from the group consisting of micronized zinc oxide surface-treated with an alkoxysilane; micronized titanium dioxide surface-treated with alkoxysilane; micronized titanium dioxide surface-treated with silica, alumina and dimethicone/methicone copolymer; micronized titanium dioxide surface-treated with alumina and dimethicone/methicone copolymer; and mixtures thereof;
(d) optionally, at least one wetting agent having a log P value of greater than about 4.0; and
(d) optionally, an organic sunscreen having a log P value of greater than about 4.0.

The soap component suitable for use in the present compositions can be an alkali metal soap (e.g., sodium, potassium) or an ammonium soap of a long-chain fatty acid containing from about 8 to about 24, preferably from about 10 to about 20, carbon atoms. Preferably, the soap component is a sodium soap or a mixture of sodium and potassium soaps. The fatty acids used in making such soaps can be obtained from natural sources well-known to persons of skill in the soap-making art. These include, without limitation, plant or animal-derived glycerides (e.g., palm oil, palm kernel oil, coconut oil, babassu oil, peanut oil, olive oil, soybean oil, castor oil, fish oil, tallow, grease, lard, or mixtures thereof). The fatty acids can also be prepared synthetically. Alkali metal soaps can be made by techniques well-known in the art (e.g., direct saponification of the fats and oils or neutralization of the free fatty acids).

As is well-known in the soap making art, soap bars are commonly made from mixtures of fatty acids. Preferred alkali metal soaps suitable for use in the present invention are the sodium and potassium salts of mixtures of fatty acids derived from coconut oil and tallow. For example, soaps of the present invention can be made from from mixtures of from about 10% to about 89% (wt/wt) of tallow fatty acid and from about 89% to 10% (wt/wt) coconut oil fatty acid. More preferably, from about 20% to about 79% tallow fatty acid is mixed with from about 79% to about 20% coconut oil fatty acid.

Red petrolatum as used in the present invention is a non-synthetic, non-irritating, non-allergenic, moisturizing ingredient, comprising branched and unbranched hydrocarbons. More particularly, it is a mixture of paraffin, isoparaffin and cycloparaffin. In soap bars of the present invention, red petrolatum is used at concentrations greater than 10% and less than about 25%. Preferably, red petrolatum is used at concentrations of from about 11% to about 20%, and more preferably at concentrations of from about 11% to about 15%.

Surface-treated metal oxide sunscreens suitable for use in the present invention are selected from the group consisting of micronized zinc oxide pigments surface-treated with alkoxysilane; micronized titanium dioxide pigments surface-treated with alkoxysilane; micronized titanium dioxide pigments surface-treated with silica, alumina and dimethiconelmethicone copolymer; micronized titanium dioxide pigments surface-treated with alumina and dimethicone/methicone copolymer; and mixtures thereof. Each of these metal oxide sunscreens is commercially available from BASF. Uvinul brand micronized titanium dioxide pigments are surface-treated with an alkoxysilane, specifically trimethoxycaprylylsilane. Z-Cote brand micronized zinc oxide pigments are also surface-treated with an alkoxysilane, triethoxycaprylylsilane. Both of these micronized metal oxide pigments, and their uses in sunscreen products, are further described in U.S. Pat. No. 5,223,250, U.S. Pat. No. 5,536,492 and U.S. Pat. No. 5,556,591, the disclosures of which are incorporated herein by reference. T-Lite brand micronized titanium dioxide pigments are surface-treated with (i) silica, alumina and dimethiconelmethicone copolymer or (ii) alumina and dimethicone/methicone copolymer.

In embodiments of the present invention comprising as a metal oxide sunscreen micronized TiO₂ pigment surface-treated with alkoxysilane, this sunscreen is present at concentrations of from about 0.5% to about 25.0%, preferably from about 1% to about 10%, more preferably from about 1% to about 5% and still more preferably less, than about 4%. The less than about 4% upper limit is important in order to achieve a desired aesthetic property of a particularly preferred embodiment of the present invention—one where a residual sunscreen film left after washing is substantially invisible. By “washing” is meant wetting the claimed soap bar with water, rubbing the wetted soap bar on a substrate to form lather, and thereafter rinsing the lather off with water. As used in the present invention, a “substantially invisible” residual film is one that after washing exhibits a level of opacity that is not readily detected by the human eye.

Micronized TiO₂ pigments surface-treated with (i) silica, alumina and dimethicone/methicone copolymer or (ii) alumina and dimethiconelmethicone copolymer may be used in soap bars of the present invention at concentrations of from about 0.5% to about 25%, preferably from about 1% to about 10% and more preferably from about 2% to about 8%, and still more preferably from about 3% to about 5%. In the preferred, more preferred, and still more preferred embodiments described above (i.e., where micronized TiO₂ pigments surface-treated with (i) silica, alumina and dimethicone/methicone copolymer or (ii) alumina and dimethicone/methicone copolymer are used at concentrations of less than about 10%, less than about 8%, and less than about 5%), the residual sunscreen film left after washing is substantially invisible.

Micronized ZnO surface-treated with alkoxysilane may be used in soap bars of the present invention at concentrations of from about 5% to about 25%, more preferably from about 10% to about 20% and most preferably from about 13% to about 17%.

Surprisingly, the inventors have found that the 10% minimum concentration of red petrolatum is critical to the ability of the present invention to effectively deposit a film containing the claimed metal oxide sunscreens after washing with the claimed soap bar.

Optional wetting agent(s) suitable for use in the present invention have a Log P of greater than about 4.0. Log P is a measure of differential solubility of a compound in two solvents. The log ratio of the concentrations of the solute in the solvent is called Log P or the Partition Coefficient. The most well-known of these partition coefficients is based on the solvents Octanol and Water. Preferably, the optional wetting agent is selected from the group consisting of hydrocarbons, mineral oils, animal or vegetable oils, silicone oils, and mixtures thereof. Non-limiting examples of optional wetting agents suitable for use in the present invention include cyclomethicone (Silicone 245 Fluid from Dow Corning; Log P˜5.7), soybean oil (Log P˜7.3), canola oil (Log P˜7.5). 20% alpha-tocopherol (vitamin E) in a solution of grapeseed and almond oils (Log P˜8.3) has been found to be a particularly effective wetting agent for the metal oxides used in soap bars of the present invention. This Vitamin E wetting agent is available commercially as Callisto Vitamin E Oil (Yasoo Health, Johnson City, Tenn.). Octocrylene (Log P˜6.9) has also been found to be an effective wetting agent for the metal oxides of the present invention.

Optionally, one or more organic sunscreens having a log P of greater than about 4.0 may be added to soap composition of the present invention. Non-limiting examples of organic sunscreens meeting this criterion are octylmethoxycinnamate, octocrylene, octyl salicylate. A preferred organic sunscreen for inclusion in compositions of the present invention is octocrylene. Protection from ultraviolet radiation is determined by methodologies well-known to persons of skill in the art. These include, without limitation, the methods described in Wendell et. al “A new in vitro test method to assess the UVA protection performance of sun care products” SÖFW-Journal 127 11-2001 and Sayre et al. “Sunscreen testing methods: in vitro predictions of effectiveness” J. Soc. Cosmet. Chem. 31 (1980) 133-143.

In another embodiment of the invention, insect repellants are included in the soap-sunscreen bar of the present invention. In a preferred embodiment, in combination with a silicone oil wetting agent (e.g. Dow Corning Silicone Fluid 245), N,N-diethyl-m-toluamide, commonly known as DEET (sold by Morflex, Inc.), can be incorporated into soap bars of the present invention. Natural insect repellents, including but not limited to, soybean oil can be used also be used in soap-sunscreen bar of the present invention.

The composition of the present invention may contain one or more diluents well-known to persons of ordinary skill in the art including, but not limited to, rheology modifiers, emulsifiers, pH modifiers, moisturizers (e.g., aloe extract), humectants, emollients (e.g., caprylic/capric triglycerides), structuring agents (e.g., beeswax, candelilla wax, paraffin), stabilizers, lubricants, fragrances, preservatives (e.g., propylparaben), colored pigments or coloring agents. Typical, non-limiting examples of diluent composition can also be found in the following U.S. patents, each of which is incorporated in its entirety by reference: U.S. Pat. Nos. 4,015,009; 4,024,106; 4,455,295; 4,613,499; 4,710,373; 4,863,963; 5,160,731; 5,338,539; 5,426,210; 5,783,173; and 5,917,088.

It is also well understood in the art that the desired plasticity of the soap bar can be achieved by including a suitable proportion of a relatively soft soap, for instance soap derived from an unsaturated fatty acid (e.g., oleic acid), or a potassium soap rather than a sodium soap. The required plasticity can also be obtained by adding a small amount of a suitable plasticizer well-known in the soap-making art.

The following examples are further illustrative of the present invention. The components and specific ingredients are presented as being typical, and various modifications can be derived in view of the foregoing disclosure within the scope of the invention.

Raw Materials

Red Petrolatum

Red Petrolatum is an item of commerce available from Penreco as 14379 Penreco Red Technical PET.

Metaloxide Sunscreens

Metaloxide sunscreens are known in the art and include:

Example 1 TiO₂ (Uvinul TiO₂, BASF).

Example 2 ZnO (Z-Cote HP1, BASF).

Organic Sunscreens

Organic sunscreens are known in the art and include:

Example 3 Octocrylene

Example 4 Octylmethoxycinnamate

Wetting Agents

Wetting agents having a Log P of greater than 4.0 are known in the art and include:

Example 5 Soybean Oil

Example 6 Cyclomethicone (Silicone 245 Fluid, Dow Corning).

Soap Bars of the Present Invention

A soap base is prepared by reacting a blend of 15% (wt/wt) tallow fatty acid and 15% (wt/wt) coconut oil fatty acid with sodium hydroxide, producing “neat soap,” a well-known term in the soap making art. Although not required, excess NaOH from the reaction may be reduced by methods well-known in the art, including for example, adding coconut oil fatty acid. The soap mass is then dried to decrease the moisture content to about 8% to about 15%.

To the specified number of grams of the soap is added the specified number of grams of red petrolatum the specified amount of the specified one surface-treated metal oxide sunscreen, the specified amount of the specified wetting agent, and the specified amount of the specified organic sunscreen. The reaction mass is well mixed and then pressed into bar form using techniques known to those skilled in the art.

| | Soap | Red | Wetting | | Metallic Oxide | | | | Organic | |
|---|---|---|---|---|---|---|---|---|---|---|
| | Base | Petrolatum | Agent | | Sunscreen | | | | Sunscreen | |
| Example | Grams | Grams | Example | Grams | TiO₂ | ZnO | Grams | | Example | Grams |
| 7 | 88.0 | 10.0 | — | — | X | | 2.0 | 0.0 | — | — |
| 8 | 80.0 | 10.0 | 5 | 2.0 | X | X | 1.0 | 5.0 | 3 | 2.0 |
| 9 | 70.0 | 10.0 | 6 | 5.0 | | X | — | 10.0 | 3 | 5.0 |
| 10 | 70.0 | 20.0 | 5 | 0.5 | X | X | 1.5 | 8.0 | — | — |
| 11 | 70.0 | 15.0 | 6 | 5.0 | X | X | 2.0 | 7.0 | 4 | 1.0 |
| 12 | 80.0 | 10.0 | 5 | 1.0 | X | X | 2.0 | 7.0 | — | — |
| 13 | 70.0 | 20.0 | 6 | 2.0 | X | X | 1.0 | 7.0 | — | — |

The soap bars of the present invention provide cleansing and provide protection from ultraviolet radiation.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A soap bar that after the washing process leaves a residual deposit on a substrate that provides protection from ultraviolet radiation comprising:
(a) a fatty soap conforming to the following structure:
R¹—C(O)—O⁻M⁺
wherein;
R¹ is alkyl having 9 to 17 carbon atoms;
M⁺ is a counterion needed for charge balance and is selected from the group consisting of Na, K and NH₄;
(b) from greater than 10% to about 25% by weight of the total composition of red petrolatum;
(c) at least one surface-treated metal oxide sunscreen;
(d) optionally, at least one wetting agent having a log P value of greater than about 4.0; and
(e) optionally, an organic sunscreen having a log P value of greater than about 4.0.

2. A soap bar of claim 1 where the substrate is skin or hair of a mammal.

3. A soap bar of claim 1 wherein the fatty soap is a sodium soap or a mixture of sodium and potassium soaps.

4. A soap bar of claim 3 wherein the fatty soap is comprised of a tallow fatty acid, a coconut oil fatty acid, or mixtures thereof.

5. A soap bar of claim 1 wherein the red petrolatum is present at concentrations of from about 11% to about 20% by weight of the composition.

6. A soap bar of claim 1 wherein the red petrolatum is present at concentrations of from about 11% to about 15%.

7. A soap bar of claim 1 wherein the surface-treated metal oxide sunscreen is selected from the group consisting of micronized zinc oxide surface-treated with an alkoxysilane; micronized titanium dioxide surface-treated with alkoxysilane; micronized titanium dioxide surface-treated with (i) silica, alumina and dimethicone/methicone copolymer or (ii) alumina and dimethicone/methicone copolymer; and mixtures thereof.

8. A soap bar of claim 1 wherein the metal oxide sunscreen is micronized TiO₂ pigments surface-treated with alkoxysilane, said metal oxide sunscreen being present at concentrations of from about 0.5% to about 25%.

9. A soap bar of claim 1 wherein the metal oxide sunscreen is micronized TiO₂ pigments surface-treated with alkoxysilane and said metal oxide sunscreen being present at concentrations of from about 1% to about 10%.

10. A soap bar of claim 1 wherein the metal oxide sunscreen is micronized TiO₂ pigments surface-treated with alkoxysilane, said metal oxide sunscreen being present at concentrations of less than about 4%.

11. A soap bar of claim 1 wherein the metal oxide sunscreen is micronized TiO₂ pigments surface-treated with (i) silica, alumina and dimethiconelmethicone copolymer or (ii) alumina and dimethicone/methicone copolymer, said metal oxide sunscreen being present at concentrations of from about 0.5% to about 20%.

12. A soap bar of claim 1 wherein the metal oxide sunscreen is micronized TiO₂ pigments surface-treated with (i) silica, alumina and dimethicone/methicone copolymer or (ii) alumina and dimethicone/methicone copolymer, said metal oxide sunscreen being present at concentrations of from about 1% to about 10%.

13. A soap bar of claim 1 wherein the metal oxide sunscreen is micronized TiO₂ pigments surface-treated with (i) silica, alumina and dimethicone/methicone copolymer or (ii) alumina and dimethicone/methicone copolymer, said metal oxide sunscreen being present at concentrations of from about 2% to about 8%.

14. A soap bar of claim 1 wherein the metal oxide sunscreen is micronized TiO₂ pigments surface-treated with silica, alumina and dimethicone/methicone copolymer, said metal oxide sunscreen being present at concentrations of less than about 3%.

15. A soap bar of claim 1 wherein the metal oxide sunscreen is micronized zinc oxide pigments surface-treated with triethoxycaprylylsilane, said metal oxide sunscreen being present at concentrations of from about 5% to about 25%.

16. A soap bar of claim 1 wherein the metal oxide sunscreen is micronized zinc oxide pigments surface-treated with triethoxycaprylylsilane, said metal oxide sunscreen being present at concentrations of from about 10% to about 20%.

17. A soap bar of claim 1 wherein the metal oxide sunscreen is micronized zinc oxide pigments surface-treated with triethoxycaprylylsilane, said metal oxide sunscreen being present at concentrations of from about 13% to about 17%.

18. A soap bar that after the washing process leaves a residual deposit on a substrate that provides protection from ultraviolet radiation comprising:
(a) a fatty soap conforming to the following structure:
R¹—C(O)—O⁻M⁺
wherein;
R¹ is alkyl having 9 to 17 carbon atoms;
M⁺ is a counterion needed for charge balance and is selected from the group consisting of Na, K and NH₄;
(b) from greater than 10% to about 25% by weight of the total composition of red petrolatum;
(c) at least one surface-treated metal oxide sunscreen selected from the group consisting of micronized zinc oxide surface-treated with an alkoxysilane; micronized titanium dioxide surface-treated with alkoxysilane; micronized titanium dioxide surface-treated with (i) silica, alumina and dimethicone/methicone copolymer or (ii) alumina and dimethicone/methicone copolymer; and mixtures thereof; and
(d) at least one wetting agent having a log P value of greater than about 4.0.

19. A soap bar of claim 1 where the substrate is skin or hair of a mammal.

20. A soap bar of claim 18 further comprising an organic sunscreen having a log P value of greater than about 4.0.

21. A soap bar of claim 1 further comprising an insect repellant.

22. A soap bar of claim 1 that after washing deposits a substantially invisible residual film containing metal oxide sunscreens.

23. A soap bar of claim 18 that after washing deposits a substantially invisible residual film containing metal oxide sunscreens.

24. A process for cleaning and providing protection to a substrate from ultraviolet radiation which comprises contacting the substrate with water and a soap bar of claim 1.

25. A process for cleaning and providing protection to a substrate from ultraviolet radiation which comprises contacting the substrate with water and a soap bar of claim 18.
